(19) United States
(12) Patent Application Publication
(10) Pub. No.: US 2024/0316141 A1
(43) Pub. Date: Sep. 26, 2024

(54) The Use of Thiourea and Thiourea Derivatives As Potentiators of Antibacterial Activity of Peptoids

(71) Applicant: The Board of Trustees of the Leland Stanford Junior University, Stanford, CA (US)

(72) Inventors: Laurent BEKALE, Redwood City, CA (US); Peter Luke SANTA MARIA, Emerald Hills, CA (US); Annelise E. BARRON, Palo Alto, CA (US); Jennifer Sue LIN, Redwood City, CA (US)

(21) Appl. No.: 18/578,153

(22) PCT Filed: Jul. 15, 2022
(86) PCT No.: PCT/US22/37295

§ 371 Date: Jan. 10, 2024

Related U.S. Application Data
(60) Provisional application No. 63/222,632, filed on Jul. 16, 2021.

(51) Int. Cl. A61K 38/08; A61K 31/17; A61K 31/5383; A61K 38/10; A61P 31/04

(52) U.S. Cl. CPC A61K 38/08; A61K 31/17; A61K 31/5383; A61K 38/10; A61P 31/04

(57) ABSTRACT

Methods for treating bacterial infections using combination therapy with thiourea or a derivative thereof and an antimicrobial peptoid are disclosed. Treatment may further comprise administration of an antibiotic. The disclosed methods may be used for the treatment of acute and chronic bacterial infections. The antimicrobial peptoid may be a self-assembly peptoid.

### BACKGROUND

Antibiotics are the mainstay of modern clinical medicine. However, bacteria develop resistance to both natural and synthetic antibiotics within years of their first clinical use (Walsh (2003) Nature Reviews Microbiology 1:65-70). Current mechanisms of antibiotic resistance include: decreased uptake by changes in outer membrane permeability; antibiotic excretion by activation of efflux pump-proteins; enzymatic modification of the antibiotic; modification of antibiotic targets; and bacterial physiology such as biofilm (van Hoek et al. (2011) Front Microbiol 2:203).

In the United States and Europe alone, over 50,000 people die every year because of resistant infections (The Review on Antimicrobial Resistance. Antimicrobial Resistance: Tackling a crisis for the health and wealth of nations (2014), amr-review.org/Publications.html)). Lengths of stays in a hospital are prolonged by antibiotic-resistant infections, and these same infections are often acquired in hospitals. The economic impact of antibiotic resistant infections is estimated to be between US $5 billion and US $24 billion per year in the United States alone (Hall (2004) Nature Reviews Microbiology 2:430-435). However, the drug pipelines of pharmaceutical companies have not kept pace with the evolution of antibiotic resistance. In 2004, only 1.5% of all the drugs in development by the world's 15 largest pharmaceutical companies were antibiotics (Smith and Coast, “The economic burden of antimicrobial resistance: why it is more serious than current studies suggest.” (2012), researchgate.net/publication/291413454). The new reality that we must face is that the pharmaceutical companies are not presently aligned for the discovery of new antibiotics. A strategy to protect our existing antibiotics is through the use of antibiotic adjuvants, compounds that enhance the activity of current drugs and minimize, and even directly block resistance (Lu et al. (2009) Proc. Natl. Acad. Sci. U.S.A. 106(12):4629-4634, Gonzalez-Bello (2017) Bioorg. Med. Chem. Lett. 27(18):4221-4228). Another strategy is the used of ant-virulence agents. These agents can circumvent antibiotic resistance by disarming pathogens of virulence factors that facilitate human disease while leaving bacterial growth pathways (Dickey et al. (2017) Nat. Rev. Drug Discov. 16(7):457-471).

Bacterial cells, attached to a surface, can aggregate to each other to form biofilms. Bacteria growing in biofilms may exhibit increased tolerance to antimicrobial agents, which it is very difficult to eliminate or substantially reduce. Biofilm bacteria have two dormant phenotypes: the viable but non-culturable (VBNC) state and the persister state. Dormant phenotypes (VBNC and persisters) allow bacteria to survive in conditions that are deadly to the rest of their genetically identical lineage. Once in biofilms, they can escape the immune system. Thus, one of the main roles of biofilm is to provide a protective habitat for persisters and VBNC by shielding them from the immune system (Lewis (2010) Microbe (Washington, D.C.) 5(10):429-437). Another property of biofilms is their capacity to be more resistant to antimicrobial agents than planktonic cells (Spoering et al. (2001) J. Bacteriol. 183(23):6746-6751). Thus, there is an ongoing and unmet need for an improved approach to treating antibiotic resistant infections.

### SUMMARY

Methods for treating bacterial infections using combination therapy with thiourea or a derivative thereof and an antimicrobial peptoid are disclosed. Treatment may further comprise administration of an antibiotic. The disclosed methods may be used for the treatment of acute and chronic bacterial infections.

In one aspect, a method of treating a bacterial infection in a subject is provided, the method comprising administering a therapeutically effective amount of thiourea or a derivative thereof in combination with a therapeutically effective amount of an antimicrobial peptoid to the subject.

In certain embodiments, the antimicrobial peptoid is a self-assembling peptoid.

In certain embodiments, the antimicrobial peptoid is H—(NLys-Nspe(p-Br)-Nspe(p-Br))₂—NH₂ (TM4), H-Ntridec-NLys-Nspe-Nspe-NLys-NH₂ (TM5), H—(NLys-Nspe-Nspe)₃-NLys-Nspe-NLys-NH₂ (TM-16), or H-Ntridec-(NLys-Nspe-Nspe(p-Br))₂-NLys-NH₂ (TM20).

In certain embodiments, the method further comprises administering a therapeutically effective amount of at least one antibiotic.

Exemplary antibiotics include, without limitation, fluoroquinolones, aminoglycosides, penicillins, tetacyclines, cephalosporins, macrolides, sulfonamides, carbapenems, ansamycins, carbacephems, carbapenems, lincosamides, monobactams, and oxazolidinones. For example, the antibiotic may include a fluoroquinolone such as ofloxacin or a derivative thereof.

In certain embodiments, the subject has a chronic or an acute infection. In some embodiments, the bacterial infection is caused by Gram-negative or Gram-positive bacteria. In some embodiments, the bacterial infection is caused by Pseudomonas aeruginosa, Staphylococcus aureus, or Klebsiella pneumoniae. In some embodiments, the subject has an infection including, without limitation, an ear infection, a cutaneous infection, a lung infection, chronic suppurative otitis media (CSOM), an infection associated with cystic fibrosis, tuberculosis, or an infection in a wound. In some embodiments, the infection is associated with formation of a bacterial biofilm in the subject. In certain embodiments, the infection comprises pathogenic bacteria that are resistant to one or more antibiotics. In some embodiments, the subject has previously been treated for the infection with one or more antibiotics that have not successfully cleared the infection. In another embodiment, the infection is an infection (e.g. Pseudomonas) in a subject who has cystic fibrosis.

In certain embodiments, the treatment eradicates all or most biofilm bacteria and planktonic bacteria. In some embodiments, the treatment eradicates all or most persister cells, which may be, for example, in a biofilm or internalized by a macrophage. In some embodiments, the persister cells that are eradicated by the treatment described herein are multidrug tolerant persister cells. Treatment may eradiate persister cells comprising either Gram-negative or Gram-positive bacteria, including, without limitation, Pseudomonas aeruginosa persister cells.

In certain embodiments, multiple cycles of treatment are administered to the subject. For example, the thiourea or derivative thereof and the antimicrobial peptoid may be administered in combination intermittently or according to a daily dosing regimen.

Compositions comprising the thiourea or derivative thereof and/or the antimicrobial peptoid may be administered by any suitable mode of administration. For example, compositions may be administered intravenously, subcutaneously, by inhalation, or topically. Alternatively, the composition may be administered locally at the site of infected tissue. For example, for an ear infection, the composition comprising the thiourea or derivative thereof and/or the antimicrobial peptoid may be administered locally into the ear canal. The thiourea or derivative thereof and the antimicrobial peptoid may be administered in the same composition or in separate compositions and may be administered by the same or different routes of administration as long as the synergistic effect of the combination treatment is achieved.

In another aspect, a composition for use in treatment of a bacterial infection is provided, the composition comprising thiourea or a derivative thereof and an antimicrobial peptoid.

In certain embodiments, the antimicrobial peptoid is a TM4 peptoid, a TM5 peptoid, a TM16 peptoid, or a TM20 peptoid.

In certain embodiments, the composition further comprises an antibiotic.

In certain embodiments, the composition further comprises a pharmaceutically acceptable excipient.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. The minimum inhibitory concentration (MIC) of peptoid (TM5) in the presence and absence of thiourea was examined. MIC was defined as the lowest concentration of peptoid that will inhibit the bacterial growth (i.e., increasing absorbance at 600 nm) after 24 h incubation. The figure shows that thiourea increases antimicrobial activity of a peptoid against Pseudomonas aeruginosa.

FIG. 2. The minimum inhibitory concentration (MIC) of peptoid (TM5) in the presence and absence of thiourea was examined. MIC was defined as the lowest concentration of peptoid that will inhibit the bacterial growth (i.e., increasing absorbance at 600 nm) after 24 h incubation. The figure shows that thiourea increases antimicrobial activity of a peptoid (TM5) against Klebsiella pneumoniae.

FIG. 3. The minimum inhibitory concentration (MIC) of peptoid (TM5) in the presence and absence of thiourea was examined. MIC was defined as the lowest concentration of peptoid that will inhibit the bacterial growth (i.e., increasing absorbance at 600 nm) after 24 h incubation. The figure shows that thiourea increases antimicrobial activity of a peptoid (TM5) against Staphylococcus aureus.

FIG. 4. To demonstrate that thiourea increases antimicrobial activity of a peptoid against PA01 ofloxacin induced persister cells, we created a population that consists solely of persister cells and has their population increased by 105-fold by treating stationary phase culture with ofloxacin (5 μg/ml) for 24 h to kill any nonpersister cells and concentrate the survival persister cells. The figure shows that thiourea enhance the persister eradication capability of TM5.

FIG. 5. Thiourea increases antimicrobial activity of a peptoid against Pseudomonas aeruginosa biofilm.

FIGS. 6A-6E. Thiourea increases antimicrobial activity of different peptoids, including TM4 (FIG. 6A), TM5 (FIG. 6B), TM16 (FIG. 6C), and TM20 (FIG. 6D). FIG. 6E shows a table comparing the minimum inhibitory concentration (MIC) of the antimicrobial peptides in the presence and absence of thiourea.

FIG. 7. The minimum inhibitory concentration (MIC) of an antimicrobial peptide (LL-37) in the presence and absence of thiourea was examined. MIC was defined as the lowest concentration of an antimicrobial peptide that will inhibit the bacterial growth (i.e., increasing absorbance at 600 nm) after a 24 hour incubation. The figure shows that thiourea did not synergize with LL-37, suggesting that synergistic combination is restricted to antimicrobial peptoids.

FIG. 8. The minimum inhibitory concentration (MIC) of peptoid (TM5) in the presence and absence of urea was examined. MIC was defined as the lowest concentration of peptoid that will inhibit the bacterial growth (i.e., increasing absorbance at 600 nm) after a 24 hour incubation. The figure shows that urea did not synergize with TM5, suggesting that synergistic combination is restricted to thiourea and thiourea derivatives.

FIGS. 9A-9E. The chemical structures of thiourea (FIG. 9A) and the peptoids, TM4 (FIG. 9B), TM5 (FIG. 9C), TM16 (FIG. 9D), and TM20 (FIG. 9E) are shown.

### DETAILED DESCRIPTION OF EMBODIMENTS

Methods for treating bacterial infections using combination therapy with thiourea or a derivative thereof and an antimicrobial peptoid are disclosed. Treatment may further comprise administration of an antibiotic. The disclosed methods may be used for the treatment of acute and chronic bacterial infections.

Before the present methods of using thiourea or a derivative thereof and an antimicrobial peptoid for treating bacterial infections are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a bacterial cell” includes a plurality of such bacterial cells and reference to “the peptoid” includes reference to one or more peptoids and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “persister cells” refers to cells that have entered a non-growing (i.e., dormant) or extremely slow-growing physiological state that renders them less susceptible or resistant to antimicrobial drugs. Such cells may “persist” after planktonic bacterial cells have been eradicated by the immune system or conventional treatment with an antimicrobial agent. Persister cells are commonly found in biofilms.

As used herein, the term “antimicrobial agent” is interchangeable with the term “antibiotic” and refers to any agent capable of having bactericidal or bacterial static effects on growth. Antibiotics include, but are not limited to, an antimicrobial peptoid, a β-lactam antibiotic, an aminoglycoside, an aminocyclitol, a quinolone, a tetracycline, a macrolide, a lincosamide, a glycopeptide, a lipopeptide, a polypeptide antibiotic, a sulfonamide, trimethoprim, chloramphenicol, isoniazid, a nitroimidazole, a rifampicin, a nitrofuran, methenamine, and mupirocin.

The term “anti-bacterial effect” means the killing of, or inhibition or stoppage of the growth and/or reproduction of bacteria.

The term “treatment” as used herein refers to (1) the prevention of infection or reinfection (prophylaxis), (2) the eradication of an existing infection, or (3) the reduction or elimination of symptoms of an infectious disease of interest (therapy).

By “therapeutically effective dose or amount” of a peptoid and thiourea (or a derivative thereof) is intended an amount that, when administered in combination, as described herein, brings about a positive therapeutic response, such as improved recovery from an infection, including any infection caused by Gram-positive or Gram-negative bacteria. Additionally, a therapeutically effective dose or amount may eradicate persister cells as well as other bacterial cells, including planktonic bacteria as well as bacteria in biofilms. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Substantially purified” generally refers to isolation of a component such as a substance (e.g., compound, peptoid, nucleic acid, polynucleotide, RNA, DNA, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “recipient”, “individual”, “subject”, “host”, and “patient”, are used interchangeably herein and refer to any vertebrate subject for whom diagnosis, treatment, or therapy is desired, particularly humans. By “vertebrate subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

The term “peptoid” refers to an oligomer comprising two or more N-substituted glycine residues. The side chain of each residue in a peptoid is connected to the amide nitrogen of the peptoid backbone, instead of the α-carbon as in peptides.

### Antimicrobial Peptoids

As explained above, the subject methods include administering an antimicrobial peptoid in combination with thiourea, or a derivative thereof. The antimicrobial peptoid for use in the methods may include peptoids known in the art to possess antimicrobial activity. By “antimicrobial activity” is meant that the peptoid is capable of having bactericidal or bacterial static effects on growth of bacteria. In some embodiments, the antimicrobial peptoid has an “anti-bacterial effect” when administered in combination with thiourea; that is, combination therapy with an antimicrobial peptoid and thiourea, or a derivative thereof, is capable of killing bacteria or inhibiting or preventing growth, proliferation, or reproduction of bacteria. Additionally, antimicrobial peptoids may disrupt the cell membrane or increase membrane permeability of a bacterial cell. In some cases, an antimicrobial peptoid may bind to bacterial DNA.

Peptoids generally are not readily degraded by proteases and therefore have the advantage of being more stable in vivo than peptides. Preferably, the peptoid is selectively cytotoxic to bacteria and shows little or no cytotoxicity in the subject undergoing treatment for an infection. In some embodiments, the peptoid has broad spectrum antimicrobial activity against Gram-positive bacteria and/or Gram-negative bacteria. In other embodiments, the peptoid has selective antimicrobial activity against a bacterial strain of interest.

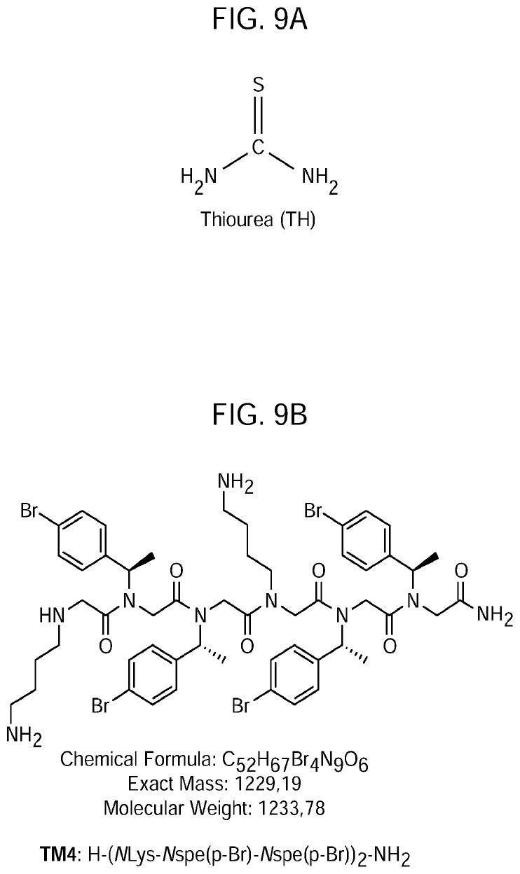

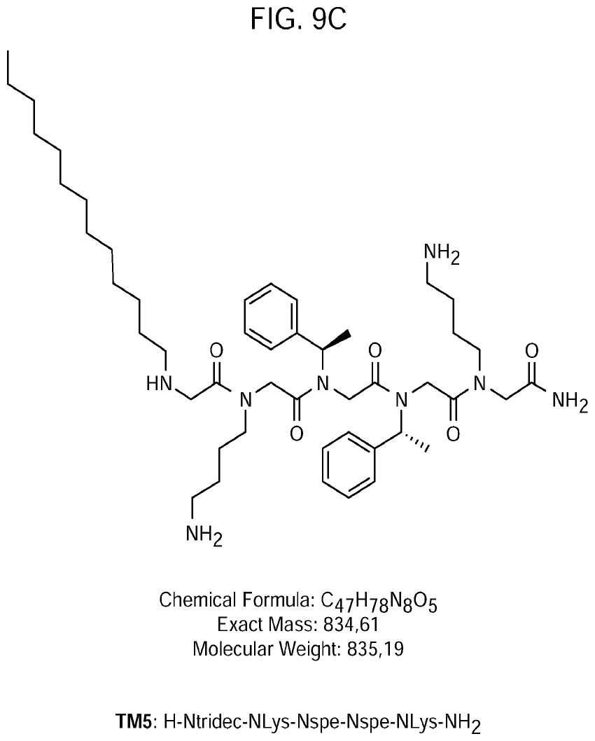

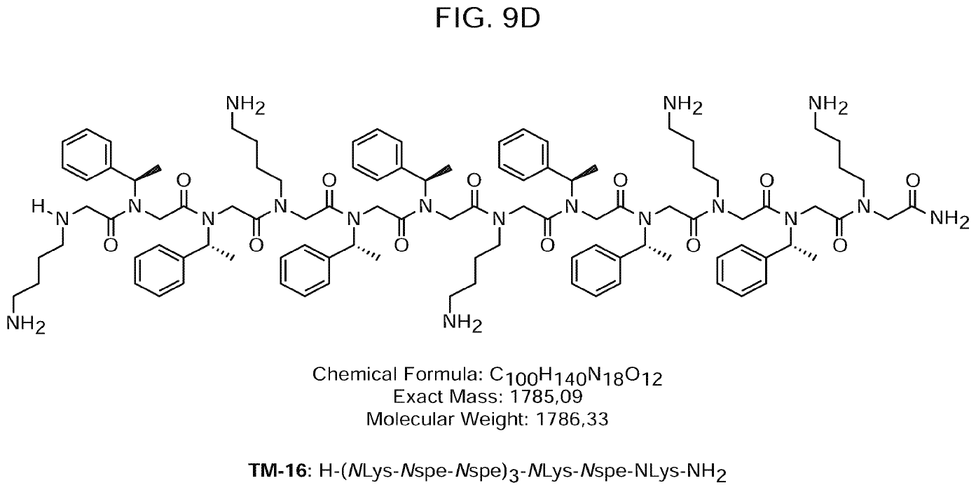

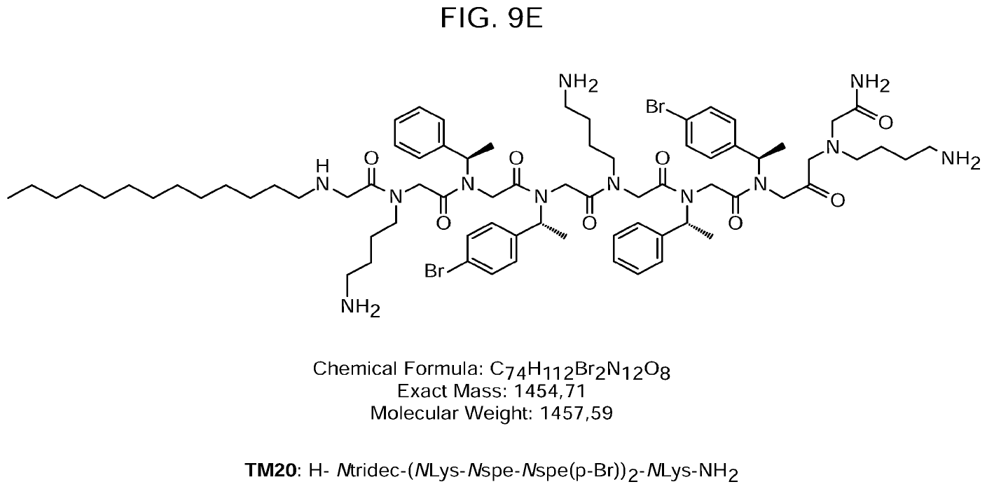

Various antimicrobial peptoids are known in the art and may be used in combination with thiourea, or a derivative thereof, for treating an infection, according to the methods described herein. FIG. 9 shows the chemical structures of a number of representative antimicrobial peptoids, including H—(NLys-Nspe(p-Br)-Nspe(p-Br))₂—NH₂ (TM4), H-Ntridec-NLys-Nspe-Nspe-NLys-NH₂ (TM5), H—(NLys-Nspe-Nspe)₃-NLys-Nspe-NLys-NH₂ (TM-16), and H-Ntridec-(NLys-Nspe-Nspe(p-Br))₂-NLys-NH₂ (TM20). Additional representative antimicrobial peptoids are described, for example, in U.S. Pat. Nos. 10,815,275; 9,315,548; U.S. Patent Application Publication No. 2008/0081789; U.S. Patent Application Publication No. 2018/0201647; U.S. Patent Application Publication No. 2015/0011465; Khara et al. (2020) Front. Microbiol. 11:417; Godballe et al. (2011) Chem. Biol. Drug Des. 77(2):107-116; Nam et al. (2020) ACS Infect Dis. 6(10):2732-2744; Hansen et al. (2013) Methods Mol Biol. 1047:151-159; Toole et al. (2021) Molecules 26(16):4741; Mojsoska (2022) Methods Enzymol. 663:327-340; Molchanova et al. (2017) Molecules 22(9):1430; and Green et al. (2020) Int. J. Antimicrob. Agents 56(2):106048; herein incorporated by reference.

In some embodiments, the peptoid self-assembles to form nanostructures in solution. Nanostructure assembly of peptoids may depend on the pH of the solution, electrostatic interactions, hydrophobic interactions, and the sequence order and side chain lengths of the peptoid. Some peptoids may form helical bundles, nanosheets, or ellipsoidal, bundled, or micellar assemblies. In some embodiments, peptoid self-assembly improves antibacterial efficacy. For a description of peptoid self-assembly, see, e.g., Knight, A. S.; Zhou, E. Y.; Francis, M. B.; Zuckermann, R. N. Sequence Programmable Peptoid Polymers for Diverse Materials Applications. Adv. Mater. 2015, 27, 5665-5691, Castelletto et al. (2020) ACS Macro Lett. 9(4):494-499, Battigelli et al. (2019) Biopolymers 10(4):e23265, Nielsen et al. (2022) ACS Infect Dis. 8(3):533-545; herein incorporated by reference.

### Pharmaceutical Compositions

Thiourea, or a derivative thereof, and an antimicrobial peptoid can be formulated into pharmaceutical compositions, optionally comprising one or more pharmaceutically acceptable excipients. The thiourea, or a derivative thereof and the antimicrobial peptoid may be combined in a single pharmaceutical composition or formulated in separate pharmaceutical compositions. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the thiourea, or derivative thereof, and the antimicrobial peptoid, or other components of the preparation. Suitable antioxidants for use include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the thiourea, or a derivative thereof, and the antimicrobial peptoid (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional compositions include those for oral, topical, transcutaneous, transdermal intratympanic, ocular, or localized delivery. Formulations suitable for topical, transcutaneous, transdermal, intratympanic, or ocular administration may be prepared through use of appropriate suspending agents, solubilizers, thickening agents, stabilizers, and preservatives. Such formulations may be utilized as liquid drops or with a means to provide continuous administration, for example, incorporation into slow-release pellets or controlled-release patches.

The pharmaceutical preparations herein can also be housed in a syringe, an implantation device, or the like, depending upon the intended mode of delivery and use. Preferably, the compositions comprising the thiourea, or derivative thereof, and the antimicrobial peptoid are in unit dosage form, meaning an amount of a composition appropriate for a single dose, in a premeasured or pre-packaged form.

The compositions herein may optionally include one or more additional agents, such as antibiotics, adjuvants, immunostimulatory agents, vaccines, and/or other medications used to treat a subject for an infection. Compounded preparations may include the thiourea, or derivative thereof, and the antimicrobial peptoid and one or more other agents for treating an infection, such as, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicillin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin; ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; quinolones/fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil, Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox, Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin, Advocid), difloxacin (Dicural, Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin(Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, sumamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I. N. H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines against bacteria and infectious diseases, including any vaccine comprising bacterial antigenic proteins or attenuated or dead bacteria and, optionally, adjuvants for boosting an immune response against bacteria, such as vaccines against tuberculosis, diphtheria, tetanus, pertussis, Haemophilus influenzae type B, cholera, typhoid, Streptococcus pneumoniae, and the like.

Alternatively, such agents can be contained in a separate composition from the composition comprising the thiourea, or derivative thereof, and the antimicrobial peptoid and co-administered concurrently, before, or after the composition comprising the thiourea, or derivative thereof, and the antimicrobial peptoid.

### Administration

At least one therapeutically effective dose of thiourea, or a derivative thereof, in combination with a therapeutically effective dose of an antimicrobial peptoid are administered. By “therapeutically effective dose or amount” of each of these agents is intended an amount that when administered in combination, brings about a positive therapeutic response with respect to treatment of an individual for an infection, including any infection caused by Gram-positive or Gram-negative bacteria. For example, a “positive therapeutic response” may include the eradication of an existing infection or the reduction or elimination of symptoms of an infectious disease. Additionally, a therapeutically effective dose or amount may eradicate persister cells and/or dormant bacteria as well as other bacterial cells, including planktonic bacteria and bacteria in biofilms.

Bacterial infections that can be treated by the methods described herein include bacterial infections caused by Gram negative bacteria such as, but not limited to, Acinetobacter (e.g., Acinetobacter baumannii), Actinobacillus, Bordetella, Brucella, Campylobacter, Cyanobacteria, Enterobacter (e.g., Enterobacter cloacae), Erwinia, Escherichia coli, Franciscella, Helicobacter (Helicobacter pylori), Hemophilus (e.g., Hemophilus influenzae), Klebsiella (e.g., Klebsiella pneumoniae), Legionella (e.g., Legionella pneumophila), Moraxella (e.g., Moraxella catarrhalis), Neisseria (e.g., Neisseria gonorrhoeae, Neisseria meningitidis), Pasteurella, Proteus (e.g., Proteus mirabilis), Pseudomonas (e.g., Pseudomonas aeruginosa), Salmonella (e.g., Salmonella enteritidis, Salmonella typhi), Serratia (e.g., Serratia marcescens), Shigella, Treponema, Vibrio (e.g., Vibrio cholerae), and Yersinia (e.g., Yersinia pestis), as well as Gram positive bacteria such as, but not limited to, Actinobacteria, such as Actinomyces (e.g., Actinomyces israelii), Arthrobacter, Bifidobacterium, Corynebacterium (e.g., Corynebacterium diphtheriae), Frankia, Micrococcus, Micromonospora, Mycobacterium (e.g., Mycobacterium tuberculosis, Mycobacterium leprae), Nocardia, Propionibacterium, and Streptomyces; Firmicutes, such as Bacilli, order Bacillales including Bacillus, Listeria (e.g., Listeria monocytogenes), and Staphylococcus (e.g., Staphylococcus aureus, Staphylococcus epidermidis), Bacilli (e.g., Bacilli anthracis, Bacilli cereus), order Lactobacillies, including Enterococcus, Lactobacillus, Lactococcus, Leuconostoc, Pediococcus, and Streptococcus (e.g., Streptococcus pneumoniae, Streptococcus mutans, Streptococcus sanguinis, Streptococcus pyogenes), Clostridia (e.g., Clostridioides difficile, Clostridium perfringens, Clostridium botulinum, Clostridium tetani, Clostridium sordellii), including Acetobacterium, Clostridium, Eubacterium, Heliobacterium, Heliospirillum, Megasphaera, Pectinatus, Selenomonas, Zymophilus, and Sporomusa, Mollicutes, including Mycoplasma (e.g., Mycoplasma pneumoniae), Spiroplasma, Ureaplasma, and Erysipelothrix.

In certain embodiments, the subject undergoing treatment with thiourea, or a derivative thereof, in combination with an antimicrobial peptoid, as described herein, has an infection including, without limitation, an ear infection, a cutaneous infection, a lung infection, a catheter-associated urinary tract infection, a gastrointestinal infection, chronic suppurative otitis media (CSOM), an infection associated with cystic fibrosis, tuberculosis, or an infection in a wound. In some embodiments, the infection is associated with formation of a bacterial biofilm in the subject. In certain embodiments, the infection comprises pathogenic bacteria that are resistant to one or more antibiotics. In some embodiments, the subject has previously been treated for the infection with one or more antibiotics that have not successfully cleared the infection. In some embodiments, the infection is a chronic infection.

In certain embodiments, multiple therapeutically effective doses of each of the thiourea, or derivative thereof and the antimicrobial peptoid will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, and so forth. For example, in some embodiments, the thiourea, or derivative thereof, and the antimicrobial peptoid will be administered twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., twice-weekly or thrice-weekly administration of a therapeutically effective dose) for one or more weekly cycles until the desired therapeutic response is achieved. The agents can be administered by any acceptable route of administration as noted herein below.

The thiourea, or derivative thereof, can be administered prior to, concurrent with, or subsequent to the antimicrobial peptoid. If provided at the same time as the antimicrobial peptoid, the thiourea, or derivative thereof, can be provided in the same or in a different composition. Thus, the two agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising thiourea, or a derivative thereof, and at least one therapeutically effective dose of a pharmaceutical composition comprising an antimicrobial peptoid according to a particular dosing regimen. Administration of the separate pharmaceutical compositions can be at the same time (i.e., simultaneously) or at different times (i.e., sequentially, in either order, on the same day, or on different days), so long as the therapeutic effect of the combination of these substances is caused in the subject undergoing therapy.

In certain embodiments, the thiourea, or a derivative thereof, is administered for a brief period prior to administration of the antimicrobial peptoid and continued for a brief period after treatment with the antimicrobial peptoid is discontinued in order to ensure that the thiourea levels are adequate in the subject during therapy with the antimicrobial peptoid. For example, the thiourea, or a derivative thereof, can be administered starting one week before administration of the first dose of the antimicrobial peptoid and continued for one week after administration of the last dose of the antimicrobial peptoid to the subject.

In other embodiments, the pharmaceutical compositions comprising the agents, such as the thiourea, or a derivative thereof, and/or the antimicrobial peptoid is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising the thiourea, or a derivative thereof, and/or the antimicrobial peptoid may be administered using the same or different routes of administration in accordance with any medically acceptable method known in the art. Suitable routes of administration include parenteral administration, such as subcutaneous (SC), intraperitoneal (IP), intramuscular (IM), intravenous (IV), or infusion, oral and pulmonary, nasal, topical, transdermal, and suppositories. Where the composition is administered via pulmonary delivery, the therapeutically effective dose is adjusted such that the soluble level of the agent, such as the thiourea, or a derivative thereof, and the antimicrobial peptoid in the bloodstream, is equivalent to that obtained with a therapeutically effective dose that is administered parenterally, for example SC, IP, IM, or IV. In some embodiments, the pharmaceutical composition comprising the thiourea, or a derivative thereof, and/or the antimicrobial peptoid is administered by IM or SC injection, particularly by IM or SC injection locally to a site of infection. In some embodiments, the thiourea, or a derivative thereof, and the antimicrobial peptoid is administered topically as drops, on a patch, or in a gel.

Factors influencing the respective amount of the various compositions to be administered include, but are not limited to, the mode of administration, the frequency of administration (i.e., daily, or intermittent administration, such as twice- or thrice-weekly), the particular type of infection or disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

### Kits

Kits may comprise one or more containers of the pharmaceutical compositions, described herein, comprising thiourea, or a derivative thereof, and/or an antimicrobial peptoid, and optionally one or more antibiotics for treating a bacterial infection. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with a pharmaceutical composition.

In addition to the above components, the subject kits may further include (in certain embodiments) instructions for practicing the subject methods (i.e., instructions for treating a bacterial infection with thiourea, or a derivative thereof, in combination with an antimicrobial peptoid, as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-24 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A method of treating a bacterial infection in a subject, the method comprising administering a therapeutically effective amount of thiourea or a derivative thereof in combination with a therapeutically effective amount of an antimicrobial peptoid to the subject.

2. The method of aspect 1, wherein the antimicrobial peptoid is a self-assembling peptoid.

3. The method of aspect 1, wherein the antimicrobial peptoid is selected from the group consisting of a TM4 peptoid, a TM5 peptoid, a TM16 peptoid, and a TM20 peptoid.

4. The method of any one of aspects 1-3, further comprising administering a therapeutically effective amount of at least one antibiotic.

5. The method of any one of aspects 1-4, wherein the subject has a chronic infection or an acute infection.

6. The method of any one of aspects 1-5, wherein the infection is an ear infection, a cutaneous infection, a lung infection, a catheter-associated urinary tract infection, or a gastrointestinal infection.

7. The method of any one of aspects 1-5, wherein the infection is associated with formation of a bacterial biofilm in the subject.

8. The method of aspect 7, wherein the biofilm is in a chronic wound in the subject.

9. The method of any one of aspects 1-8, wherein the subject has chronic suppurative otitis media (CSOM), cystic fibrosis, or tuberculosis.

10. The method of any one of aspects 1-9, wherein the infection comprises pathogenic bacteria that are resistant to one or more antibiotics.

11. The method of aspect 10, wherein the pathogenic bacteria are resistant to ciprofloxacin.

12. The method of any one of aspects 1-11, wherein the subject has previously been treated for the infection with one or more antibiotics that have not successfully cleared the infection.

13. The method of any one of aspects 1-12, wherein said treating eradicates all or most biofilm bacteria and planktonic bacteria.

14. The method of any one of aspects 1-13, wherein said treating eradicates all or most persister cells.

15. The method of any of aspects 1-14, wherein the bacterial infection is caused by Gram-negative or Gram-positive bacteria.

16. The method of any one of aspects 1-15, wherein the bacterial infection is caused by Pseudomonas aeruginosa, Staphylococcus aureus, or Klebsiella pneumoniae.

17. The method of any one of aspects 1-16 wherein multiple cycles of treatment are administered to the subject.

18. The method of any one of aspects 1-17, wherein the thiourea or derivative thereof and the antimicrobial peptoid are administered intravenously, subcutaneously, by inhalation, or topically.

19. The method of any one of aspects 1-17, wherein the thiourea or derivative thereof and the antimicrobial peptoid are administered locally at the site of infected tissue.

20. A composition for use in a method of treating a bacterial infection, the composition comprising thiourea or a derivative thereof and an antimicrobial peptoid.

21. The composition of aspect 20, wherein the antimicrobial peptoid is a self-assembling peptoid.

22. The composition of aspect 20, wherein the antimicrobial peptoid is H—(NLys-Nspe(p-Br)-Nspe(p-Br))₂—NH₂ (TM4), H-Ntridec-NLys-Nspe-Nspe-NLys-NH₂ (TM5), H—(NLys-Nspe-Nspe)₃-NLys-Nspe-NLys-NH₂ (TM-16), or H-Ntridec-(NLys-Nspe-Nspe(p-Br))₂-NLys-NH₂ (TM20).

23. The composition of any one of aspects 20-22, further comprising an antibiotic.

24. The composition of any one of aspects 20-23, further comprising a pharmaceutically acceptable excipient.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### The Use of Thiourea and Thiourea Derivatives as Potentiators of Antibacterial Activity of Peptoids

Antimicrobial peptoids are promising leads for use as novel antibiotics; however, their activity is often compromised under physiological conditions. Here, we show that the efficacy of antimicrobial peptoids can be enhanced by using thiourea and thiourea derivatives. The minimum inhibitory concentration (MIC) of antimicrobial peptoids against Pseudomonas aeruginosa, Staphylococcus aureus and ciprofloxacin-resistant Klebsiella pneumoniae significantly decreased when determined in the presence of thiourea as compared to with the antimicrobial peptoid alone. In addition, combination with thiourea enhanced the efficacy of antimicrobial peptoids against bacterial biofilms.

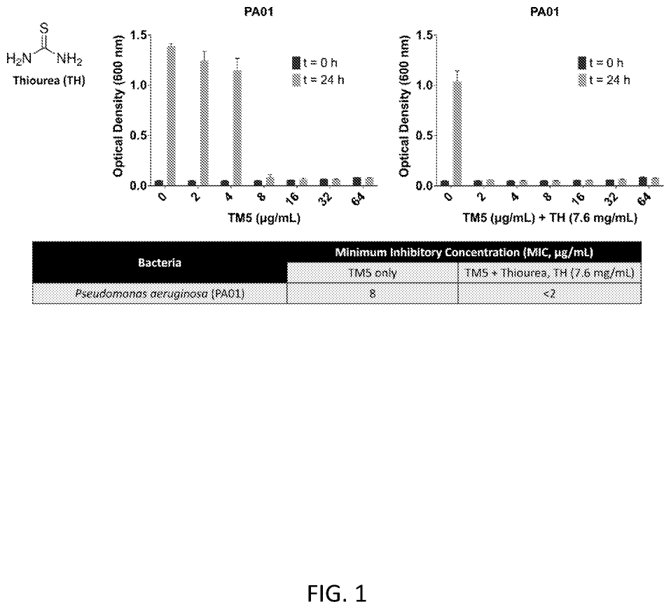

FIG. 1 shows that thiourea increased antimicrobial activity of a peptoid against Pseudomonas aeruginosa.

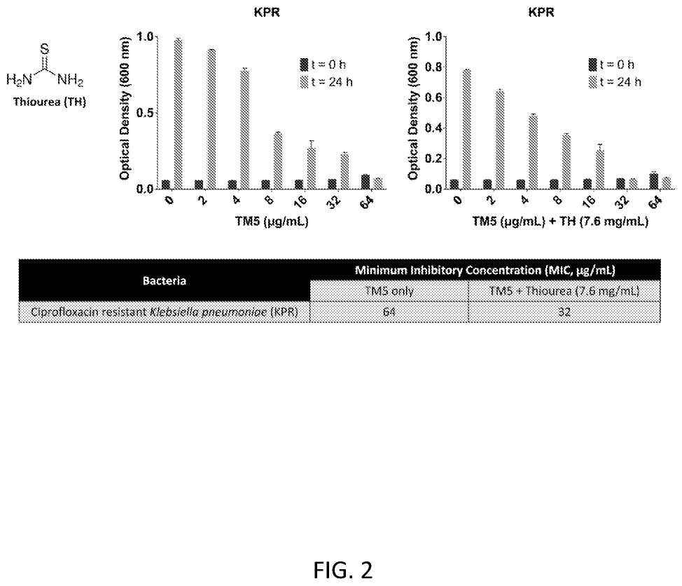

FIG. 2 shows that thiourea increased antimicrobial activity of a peptoid against Klebsiella pneumoniae.

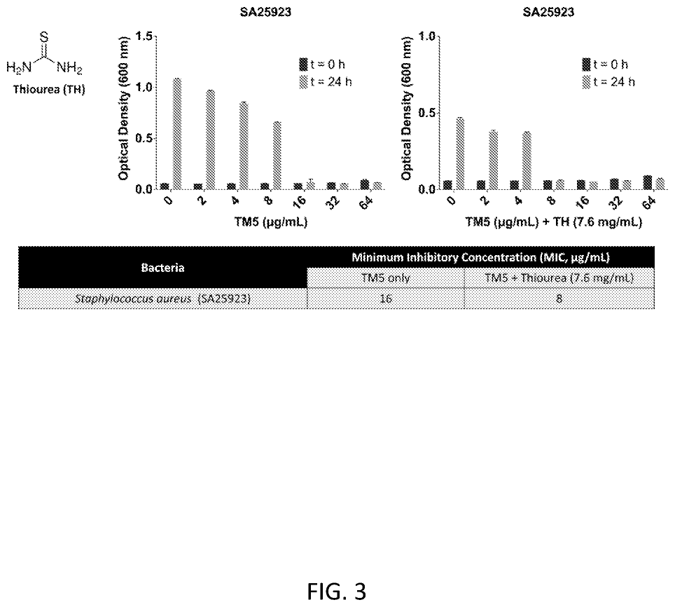

FIG. 3 show that thiourea increased antimicrobial activity of a peptoid against Staphylococcus aureus.

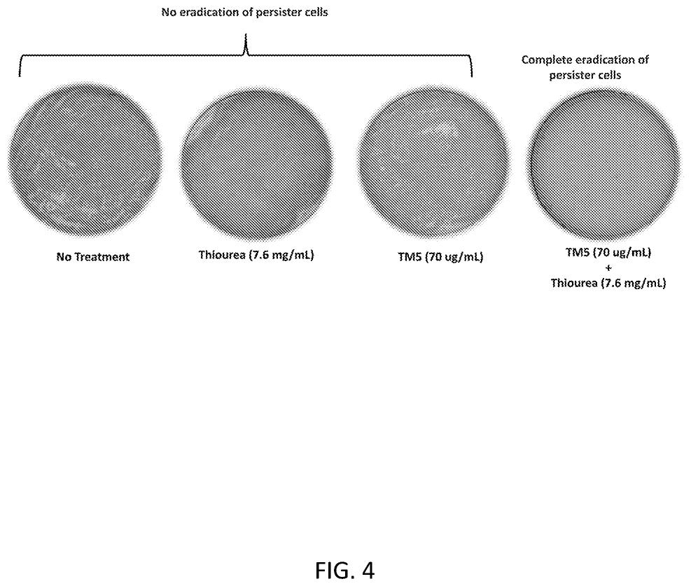

FIG. 4 shows that thiourea increased antimicrobial activity of a peptoid against PA01 ofloxacin induced persister cells.

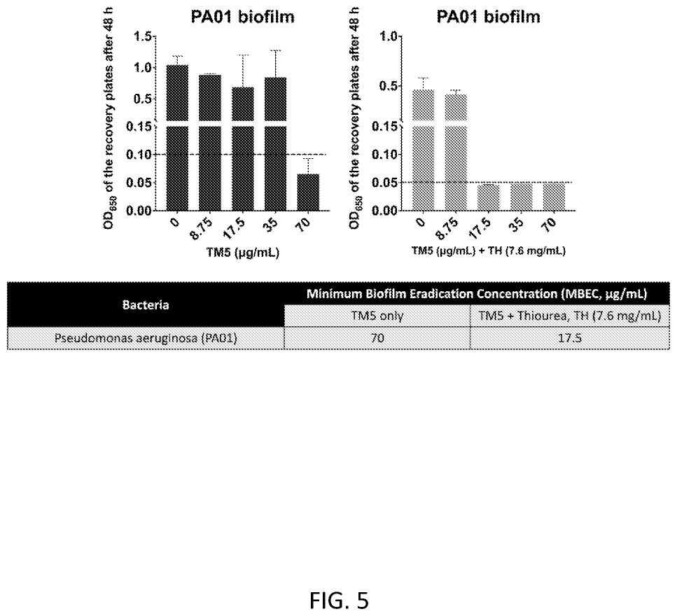

FIG. 5 shows that thiourea increased antimicrobial activity of a peptoid against Pseudomonas aeruginosa biofilm.

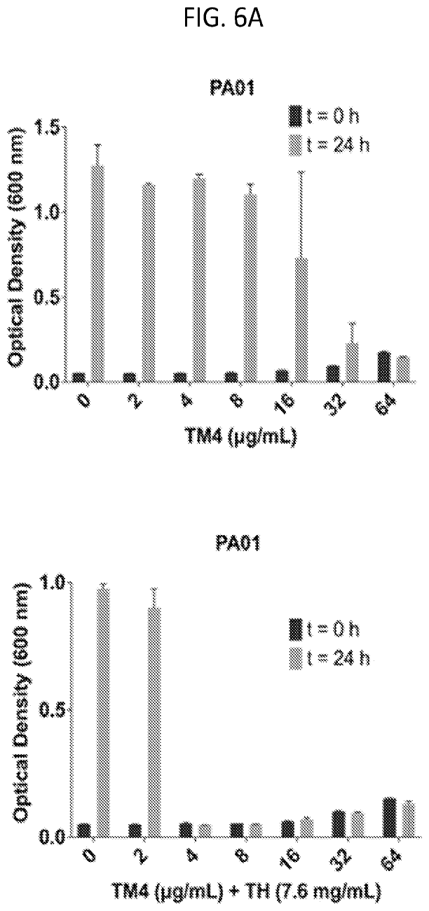

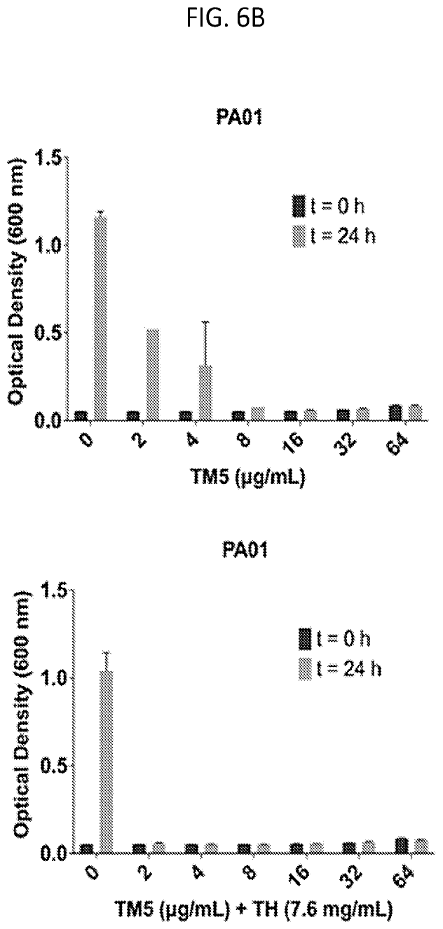

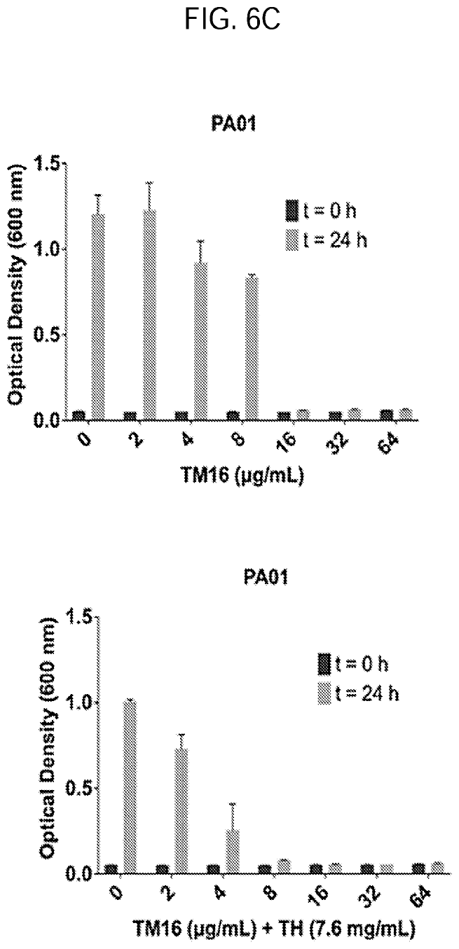

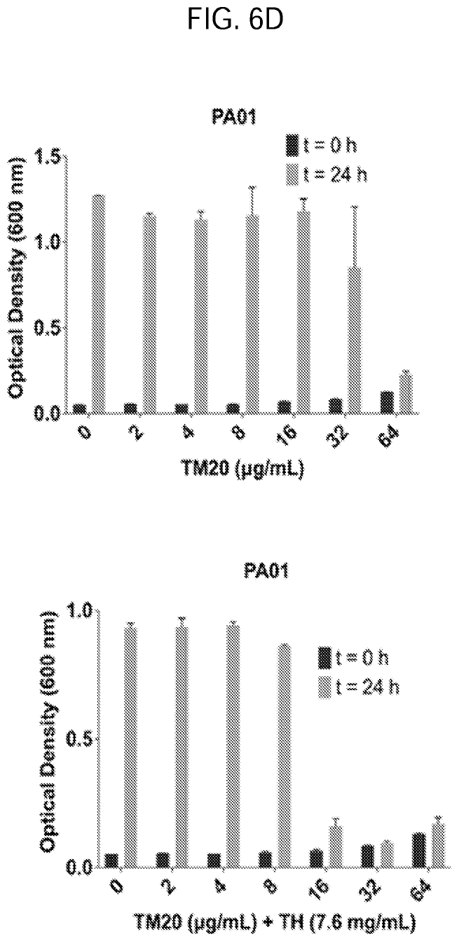

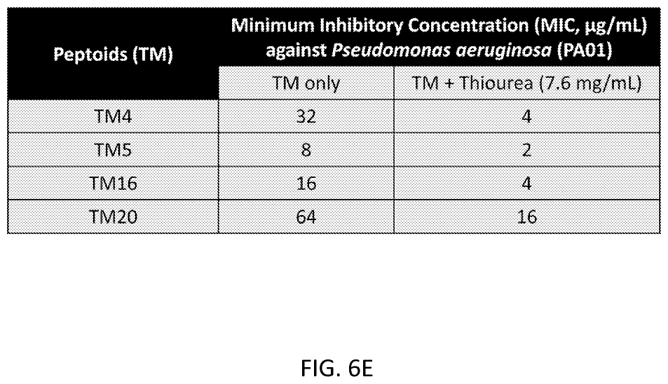

FIG. 6 shows that thiourea increased antimicrobial activity of different peptoids, including TM4, TM5, TM16, and TM20.

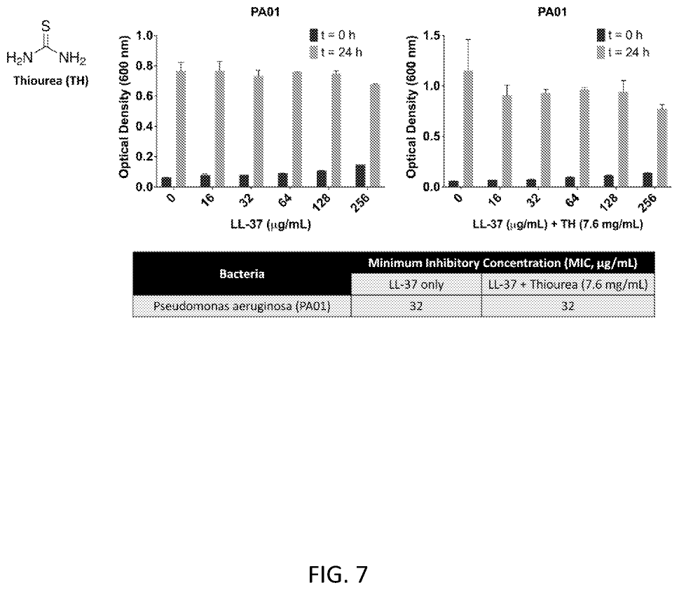

FIG. 7 shows that the synergistic combination is restricted to antimicrobial peptoids. Thiourea did not synergize with antimicrobial peptide, LL-37.

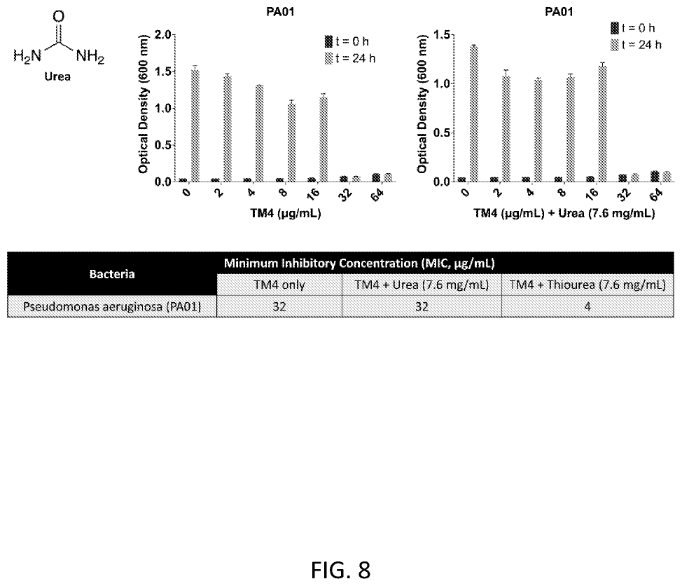

FIG. 8 shows that the synergistic combination is restricted to thiourea and thiourea derivatives. Urea did not synergize with an antimicrobial peptoid.

### APPLICATIONS

The therapeutic use of thiourea in combination with antimicrobial peptoids to treat bacterial infections could be used for the treatment of acute and chronic bacterial infections.

1. A method of treating a bacterial infection in a subject, the method comprising administering a therapeutically effective amount of thiourea or a derivative thereof in combination with a therapeutically effective amount of an antimicrobial peptoid to the subject.

2. The method of claim 1, wherein the antimicrobial peptoid is a self-assembling peptoid.

3. The method of claim 1, wherein the antimicrobial peptoid is selected from the group consisting of a TM4 peptoid, a TM5 peptoid, a TM16 peptoid, and a TM20 peptoid.

4. The method of claim 1, further comprising administering a therapeutically effective amount of at least one antibiotic.

5. The method of claim 1, wherein the subject has a chronic infection or an acute infection.

6. The method of claim 1, wherein the infection is an ear infection, a cutaneous infection, a lung infection, a catheter-associated urinary tract infection, or a gastrointestinal infection.

7. The method of claim 1, wherein the infection is associated with formation of a bacterial biofilm in the subject.

8. The method of claim 7, wherein the biofilm is in a chronic wound in the subject.

9. The method of claim 1, wherein the subject has chronic suppurative otitis media (CSOM), cystic fibrosis, or tuberculosis.

10. The method of claim 1, wherein the infection comprises pathogenic bacteria that are resistant to one or more antibiotics.

11. The method of claim 10, wherein the pathogenic bacteria are resistant to ciprofloxacin.

12. The method of claim 1, wherein the subject has previously been treated for the infection with one or more antibiotics that have not successfully cleared the infection.

13. The method of claim 1, wherein said treating eradicates all or most biofilm bacteria and planktonic bacteria.

14. The method of claim 1, wherein said treating eradicates all or most persister cells.

15. The method of claim 1, wherein the bacterial infection is caused by Gram-negative or Gram-positive bacteria.

16. The method of claim 1, wherein the bacterial infection is caused by Pseudomonas aeruginosa, Staphylococcus aureus, or Klebsiella pneumoniae.

17. The method of claim 1, wherein multiple cycles of treatment are administered to the subject.

18. The method of claim 1, wherein the thiourea or derivative thereof and the antimicrobial peptoid are administered intravenously, subcutaneously, by inhalation, or topically.

19. The method of claim 1, wherein the thiourea or derivative thereof and the antimicrobial peptoid are administered locally at the site of infected tissue.

20. A composition comprising thiourea or a derivative thereof and an antimicrobial peptoid.

21-24. (canceled)
